(19) United States
(12) Patent Application Publication
(10) Pub. No.: US 2004/0254246 A1
(43) Pub. Date: Dec. 16, 2004

(54) Treating or preventing hot flashes using prodrugs of GABA analogs

(76) Inventors: Ronald W. Barrett, Saratoga, CA (US); Mark A. Gallop, Los Altos, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/816,551

(22) Filed: Mar. 31, 2004

Related U.S. Application Data
(60) Provisional application No. 60/459,472, filed on Mar. 31, 2003.
(60) Provisional application No. 60/512,280, filed on Oct. 17, 2003.
(60) Provisional application No. 60/538,724, filed on Jan. 22, 2004.

(51) Int. Cl. A61K 31/195

(52) U.S. Cl. 514/561

(57) ABSTRACT

Disclosed herein are methods of using prodrugs of GABA analogs and pharmaceutical compositions thereof to treat or prevent hot flashes in humans and pharmaceutical compositions of prodrugs of GABA analogs useful in treating or preventing hot flashes.

This application claims the benefit under 35 U.S.C. § 119(e) from U.S. Provisional Application Ser. No. 60/459,472 filed Mar. 31, 2003; U.S. Provisional Application Ser. No. 60/512,280 filed Oct. 17, 2003; and U.S. Provisional Application Ser. No. 60/538,724 filed Jan. 22, 2004, which are herein incorporated by reference in their entirety.

### 1. TECHNICAL FIELD

The methods and pharmaceutical compositions disclosed herein relate generally to treating or preventing hot flashes in a patient. More specifically, disclosed herein are methods of using prodrugs of GABA analogs and pharmaceutical compositions thereof to treat or prevent hot flashes in patients and pharmaceutical compositions of prodrugs of GABA analogs useful in treating or preventing hot flashes.

### 2. BACKGROUND

Hot flashes or flushing occur commonly in menopausal and post-menopausal women and is characterized by a sudden onset of warmth in the chest and often progressing to the face and neck. Such episodes generally lasts several minutes and are evidenced by a visible flushing of the skin. Often such episodes are accompanied by sweating, dizziness, nausea, palpitations and diaphoresis. Such symptoms can disrupt sleep and interfere with the quality of life. Although the cause of hot flashes are not completely understood, they may be a disorder of thermoregulation resulting from a transient lowering of the hypothalamic temperature regulatory set point (Kronenberg et al., Can. J. Physiol. Pharmacol. 1987, 65, 1312-1324; Shanafelt et al., Mayo Clin. Proc. 2002, 77, 1207-1218). In post-menopausal woman, the cause of such hot flashes may be a consequence of declining estrogen levels since hot flashes also occur in women taking anti-estrogen drugs such as tamoxifen. Men also experience hot flashes following androgen-ablation therapy (from bilateral orchiectomy or treatment with a gonadotrophin-releasing-bormone agonist) for metastatic prostate cancer (Kouriefs et al., British J. Urol. Int. 2002, 89, 379-383).

Although estrogen replacement therapy is the most direct and effective treatment for hot flashes in women, there are women for whom such therapy is contraindicated (e.g., women with breast cancer or a strong family history of breast cancer, a history of clotting, severe migraine, or who are averse to taking the drug). Alternative medications exist to prevent or treat the serious consequences of menopause, such as osteoporosis and raised serum lipid levels in women averse to direct estrogen replacement therapy. Included in this category are the selective estrogen-receptor modulators (SERMs), such as raloxifene (see Cullinan, U.S. Pat. No. 5,534,526), which selectively bind to and activate the estrogen receptors of some tissues such as bone, and block the receptors of others, i.e., breast and uterus. Accordingly, many of these modulators lack the negative impact that prolonged estrogen therapy may have on these organs. However, in contrast to estrogen, SERMs are not as effective in preventing hot flashes.

Other than estrogen-replacement therapy, few effective means exist to alleviate hot flashes. Low dose oral megestrol acetate (Loprinzi et al., N. Engl. J. Med. 1994, 331, 347-351), venlafaxine (Loprinzi et al., Lancet 2000, 356, 2059-2063; Quella et al., J. Urol. 1999, 162, 98-102), transdermal clonidine, a centrally active α-agonist (Goldberg et al., J. Clin. Onc. 1994, 12, 155-158), and a variety of herbal remedies, (Shanafelt et al., Mayo Clin. Proc. 2002, 77, 1207-1218) have been used to treat hot flashes in both male and female patients.

Several recent clinical studies have suggested that the γ-aminobutyric acid (γ-aminobutyric acid is abbreviated herein as “GABA”) analog gabapentin (1) is effective in reducing the frequency and severity of hot flashes in female and male patients (Guttuso, Neurology 2000, 54, 2161-2163; Loprinzi et al., Mayo Clin. Proc. 2002, 77, 1159-1163; Jeffery et al., Ann. Pharmacother. 2002, 36, 433435; Guttuso et al., Obstet. Gynecol. 2003, 101, 337-345). Subjects treated in these studies include post-menopausal women, women with a history of breast cancer, women who have undergone hysterectomies and men receiving gonadotropin hormone-releasing hormone therapy and/or anti-androgen therapy for treatment of prostate cancer. A double-blind placebo controlled trial of gabapentin which was conducted using 59 postmenopausal women demonstrated substantial reduction in hot flash frequency from baseline (Guttuso et al., Obstet. Gynecol. 2003, 101, 337-345).

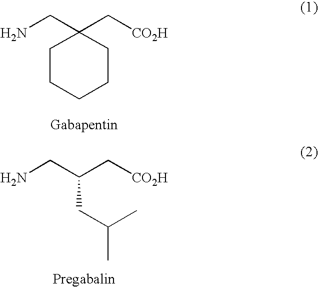

Gabapentin has been approved in the United States for the treatment of epileptic seizures and post-herpetic neuralgia. The drug has also shown efficacy in controlled studies for treating neuropathic pain of varying etiologies, as well as depression, anxiety, psychosis, faintness attacks, hypokinesia, cranial disorders, neurodegenerative disorders, panic disorders, inflammatory disease, insomnia, gastrointestinal disorders, urinary incontinence and ethanol withdrawal syndrome (Magnus, Epilepsia 1999, 40, S66-72). The broad pharmaceutical activities of GABA analogs such as gabapentin has stimulated intensive interest in preparing related compounds which have superior pharmaceutical properties in comparison to GABA, e.g., the ability to cross the blood brain barrier (see, e.g., Satzinger et al., U.S. Pat. No. 4,024,175; Silverman et al., U.S. Pat. No. 5,563,175; Horwell et al., U.S. Pat. No. 6,020,370; Silverman et al., U.S. Pat. No. 6,028,214; Horwell et al., U.S. Pat. No. 6,103,932; Silverman et al., U.S. Pat. No. 6,117,906; Silverman, International Publication No. WO 92/09560; Silverman et al., International Publication No. WO 93/23383; Horwell et al., International Publication No. WO 97/29101, Horwell et al., International Publication No. WO 97/33858; Horwell et al., International Publication No. WO 97/33859; Bryans et al., International Publication No. WO 98/17627; Guglietta et al., International Publication No. WO 99/08671; Bryans et al., International Publication No. WO 99/21824; Bryans et al., International Publication No. WO 99/31057; Belliotti et al., International Publication No. WO 99/31074; Bryans et al., International Publication No. WO 99/31075; Bryans et al., International Publication No. WO 99/61424; Bryans et al., International Publication No. WO 00/15611; Bellioti et al., International Publication No. WO 00/31020; Bryans et al., International Publication No. WO 00/50027; and Bryans et al., International Publication No. WO 02/00209). One analog of particular interest is pregabalin (2), which possesses greater potency in pre-clinical models of pain and epilepsy than gabapentin and is presently in Phase III clinical trials.

Though the mechanism of action of gabapentin in modulating these aforementioned disease states (including hot flashes) is not understood with certainty, gabapentin, pregabalin and related analogs are known to interact with the α₂δ subunit of neuronal voltage-gated calcium channels (Gee et al., J. Biol. Chem. 1996, 271, 5768-5776; Bryans et al., J. Med. Chem. 1998, 41, 1838-1845). Guttuso has described a method for treating hot flashes in a patient by administering to the patient a compound which binds an α₂δ subunit of a voltage-gated calcium channel. Preferred compounds include the GABA analogs gabapentin and pregabalin (Guttuso, U.S. Pat. No. 6,310,098).

One significant problem associated with the clinical use of many GABA analogs, including gabapentin and pregabalin, is rapid systemic clearance. Consequently these drugs require frequent dosing to maintain a therapeutic or prophylactic concentration in the systemic circulation (Bryans et al., Med. Res. Rev. 1999, 19, 149-177). For example, dosing regimens of 300-600 mg doses of gabapentin administered three times per day are typically used for anticonvulsive therapy. Higher doses (1800-3600 mg/day in three or four divided doses) are typically used for the treatment of neuropathic pain states.

Although oral sustained released formulations are conventionally used to reduce the dosing frequency of drugs that exhibit rapid systemic clearance, oral sustained release formulations of gabapentin and pregabalin have not been developed because these drugs not absorbed via the large intestine. Rather, these compounds are typically absorbed in the small intestine by one or more amino acid transporters (e.g. the “large neutral amino acid transporter,” see Jezyk et al., Pharm. Res. 1999, 16, 519-526). The limited residence time of both conventional and sustained release oral dosage forms in the proximal absorptive region of the gastrointestinal tract necessitates frequent daily dosing of conventional oral dosage forms of these drugs, and has prevented the successful application of sustained release technologies to these drugs.

One method for overcoming the problem of rapid systemic clearance of a GABA analog relies upon the administration of an extended release dosage formulation containing a GABA analog prodrug of the type disclosed by Gallop et al., in International Publication Nos. WO 02/100347 and WO 02/100349. Such prodrugs are capable of being absorbed over wider regions of the gastrointestinal tract than the parent drug, and are capable of being absorbed across the wall of the colon where sustained release oral dosage forms typically spend a significant portion of their GI transit time. These prodrugs are converted to the parent GABA analog upon absorption in vivo.

Currently available therapeutic agents for treating or preventing hot flashes have either serious side effects or reduced effectiveness. Therefore, there is a need in the art for a method of delivering an agent such as a prodrug of a GABA analog, particularly in extended release dosage form, which can treat or prevent hot flashes with a reduced risk of side effects.

### 3. SUMMARY

Methods of treating or preventing hot flashes in a patient are disclosed herein. The methods are useful in treating or preventing hot flashes in both male and female patients and are particularly useful in treating or preventing hot flashes in menopausal and post-menopausal human females.

In one aspect, a method of treating or preventing hot flashes in a patient which comprises administering to the patient a therapeutically effective amount of a prodrug of a GABA analog or a pharmaceutically acceptable salt, hydrate, solvate or N-oxide thereof is provided.

In a second aspect, a method of treating or preventing hot flashes in a patient comprising administering to the patient a pharmaceutical composition which comprises a therapeutically effective amount of a prodrug of a GABA analog or a pharmaceutically acceptable salt, hydrate, solvate or N-oxide thereof and a pharmaceutically acceptable vehicle is provided.

It should be understood that the methods and pharmaceutical compositions disclosed herein are not restricted to particular prodrugs of GABA analogs. Accordingly, the disclosed methods may be practiced with any GABA analog prodrug. A preferred class of GABA analog prodrugs are those which bind the α₂δ subunit of a voltage-gated calcium channel. Of these, prodrugs of gabapentin and pregabalin are preferred.

In one embodiment, a prodrug of a GABA analog has the structure of Formula (I):

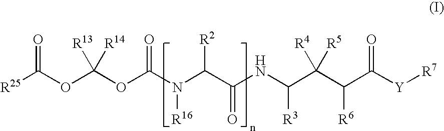

or a pharmaceutically acceptable salt, hydrate, solvate or N-oxide thereof, wherein:

n is 0 or 1;

Y is O or S;

R¹⁶ is hydrogen, alkyl or substituted alkyl;

R² is selected from the group consisting of hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, or optionally, R² and R¹⁶ together with the atoms to which they are attached form a cycloheteroalkyl or substituted cycloheteroalkyl ring;

R³ and R⁶ are independently selected from the group consisting of hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl and substituted heteroarylalkyl;

R⁴ and R⁵ are independently selected from the group consisting of hydrogen, alkyl, substituted alkyl, acyl, substituted acyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl and substituted heteroarylalkyl or optionally, R⁴ and R⁵ together with the carbon atom to which they are attached form a cycloalkyl, substituted cycloalkyl, cycloheteroalkyl, substituted cycloheteroalkyl or bridged cycloalkyl ring;

R⁷ is selected from the group consisting of hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl and substituted heteroarylalkyl;

R¹³ and R¹⁴ are each independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl, substituted heteroaryl, heteroarylalkyl or substituted heteroarylalkyl or optionally, R¹³ and R¹⁴ together with the carbon atom to which they are attached form a cycloalkyl, substituted cycloalkyl, cycloheteroalkyl or substituted cycloheteroalkyl ring; and

R²⁵ is selected from the group consisting of acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl and substituted heteroarylalkyl.

In a third aspect, there is provided a pharmaceutical composition for treating a patient suffering from hot flashes. The pharmaceutical composition comprises a therapeutically effective amount of a prodrug of a GABA analog or a pharmaceutically acceptable salt, hydrate, solvate or N-oxide thereof, and a pharmaceutically acceptable vehicle.

In a fourth aspect, there is provided a pharmaceutical composition for preventing hot flashes in a patient at a risk of hot flashes. The pharmaceutical composition comprises a therapeutically effective amount of a prodrug of a GABA analog or a pharmaceutically acceptable salt, hydrate, solvate or N-oxide thereof and a pharmaceutically acceptable vehicle.

### 4. DETAILED DESCRIPTION

### 4.1 Definitions

“Compounds” refers to GABA analogs including any compounds encompassed by generic formulae disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. In general, all physical forms are equivalent for the uses contemplated herein and are intended to be within the scope of the present invention. Further, it should be understood, when partial structures of the compounds are illustrated, that brackets indicate the point of attachment of the partial structure to the rest of the molecule.

“Alkyl” by itself or as part of another substituent refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 20 carbon atoms, more preferably, from 1 to 10 carbon atoms. (C₁-C₆) alkyl, for example, refers to an alkyl group containing from 1 to 6 carbon atoms.

“Alkanyl” by itself or as part of another substituent refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein. Representative examples include, but are not limited to formyl, acetyl, cyclohexylcarbonyl, cyclohexylmethylcarbonyl, benzoyl, benzylcarbonyl and the like.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —OR³² where R³² represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Preferably, an aryl group comprises from 6 to 20 carbon atoms, more preferably from 6 to 12 carbon atoms.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. Preferably, an arylalkyl group is (C₆-C₃₀) arylalkyl, e.g., the alkanyl, alkenyl or alkynyl moiety of the arylalkyl group is (C₁-C₁₀) and the aryl moiety is (C₆-C₂₀), more preferably, an arylalkyl group is (C₆-C₂₀) arylalkyl, e.g., the alkanyl, alkenyl or alkynyl moiety of the arylalkyl group is (C₁-C₈) and the aryl moiety is (C₆-C₁₂).

“AUC” is the area under the plasma drug concentration-versus-time curve extrapolated from zero time to infinity.

“Bridged cycloalkyl” refers to a radical selected from the group consisting of

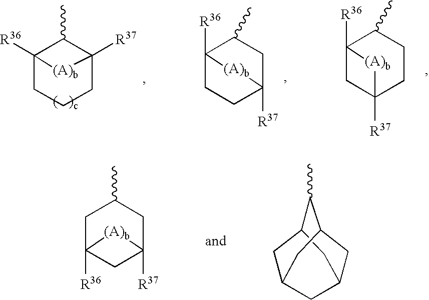

wherein:

A is (CR³⁸R³⁹)_{b};

R³⁸ and R³⁹ are independently selected from the group consisting of hydrogen and methyl;

R³⁶ and R³⁷ are independently selected from the group consisting of hydrogen and methyl;

b is an integer from 1 to 4; and

c is an integer from 0 to 2.

“Carbamoyl” by itself or as part of another substituent refers to the radical —C(O)NR⁴⁰R⁴¹ where R⁴⁰ and R⁴¹ are independently hydrogen, alkyl, cycloalkyl or aryl as defined herein.

“Cₘₐₓ” is the highest drug concentration observed in plasma following an extravascular dose of drug.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. Preferably, the cycloalkyl group is (C₃-C₁₀) cycloalkyl, more preferably (C₃-C₇) cycloalkyl.

“Cycloheteroalkyl” by itself or as part of another substituent refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine, and the like.

“GABA analog” refers to a compound, unless specified otherwise, as having the following structure:

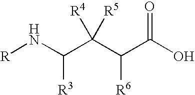

wherein:

R is hydrogen, or R and R⁶ together with the atoms to which they are attached form an azetidine, substituted azetidine, pyrrolidine or substituted pyrrolidine ring;

R³ and R⁶ are independently selected from the group consisting of hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl and substituted heteroarylalkyl; and

R⁴ and R⁵ are independently selected from the group consisting of hydrogen, alkyl, substituted alkyl, acyl, substituted acyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl and substituted heteroarylalkyl or optionally, R⁴ and R⁵ together with the carbon atom to which they are attached form a cycloalkyl, substituted cycloalkyl, cycloheteroalkyl, substituted cycloheteroalkyl or bridged cycloalkyl ring.

“Hot flashes” refer to vasomotor events characterized by the sudden onset of intense warmth that may begin in the chest and may progress to the neck and face. They are often accompanied with anxiety, palpitations, profuse sweating, and red blotching of the skin. Hot flash symptoms can adversely affect a patient's ability to work, sleep, and their general perception of health.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —O—O—, —S—S—, —O—S—, —NR⁴²R⁴³, —N—N═—, —N═N—, —N═N—NR⁴⁴R⁴⁵, —PR⁴⁶—, —P(O)₂—, —POR⁴⁷—, —O—P(O)₂—, —SO—, —SO₂—, —SnR⁴⁸R⁴⁹— and the like, where R⁴², R⁴³, R⁴⁴, R⁴⁵, R⁴⁶, R⁴⁷, R⁴⁸ and R⁴⁹ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is from 5-20 membered heteroaryl, more preferably from 5-10 membered heteroaryl. Preferred heteroaryl groups are those derived from thiophene, pyrrole, benzothiophene, benzofuran, indole, pyridine, quinoline, imidazole, oxazole and pyrazine

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In preferred embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl, more preferably, 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” refers to a parent aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” refers to a mammal, which is preferably human.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound of the invention is administered.

“Patient” includes humans. The terms “human” and “patient” are used interchangeably herein.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Prodrug” refers to a derivative of a drug molecule that requires a transformation within the body to release the active drug. Prodrugs are frequently, although not necessarily, pharmacologically inactive until converted to the parent drug. A hydroxyl containing drug may be converted to, for example, to a sulfonate, ester or carbonate prodrug, which may be hydrolyzed in vivo to provide the hydroxyl compound. An amino containing drug may be converted, for example, to a carbamate, amide, enamine, imine, N-phosphonyl, N-phosphoryl or N-sulfenyl prodrug, which may be hydrolyzed in vivo to provide the amino compound. A carboxylic acid drug may be converted to an ester (including silyl esters and thioesters), amide or hydrazide prodrug, which be hydrolyzed in vivo to provide the carboxylic acid compound. Prodrugs for drugs which have functional groups different than those listed above are well known to the skilled artisan.

“Promoiety” refers to a form of protecting group that when used to mask a functional group within a drug molecule converts the drug into a prodrug. Typically, the promoiety will be attached to the drug via bond(s) that are cleaved by enzymatic or non-enzymatic means in vivo.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Substituted” refers to a group in which one or more hydrogen atoms are independently replaced with the same or different substituent(s). Typical substituents include, but are not limited to, -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)20-, —S(O)₂OH, —S(O)₂R⁶⁰, —OS(O₂)O—, —OS(O)₂R⁶⁰, —P(O)(O—)₂, —P(O)(OR⁶⁰)(O—), —OP(O)(OR⁶⁰)(OR⁶¹), —C(O)R6, —C(S)R60, —C(O)OR⁶⁰, —C(O)NR⁶OR⁶⁰, —C(O)O⁻, —C(S)OR⁶⁰, —NR⁶²C(O)NR⁶⁰R⁶¹, —NR⁶¹C(S)NR⁶⁰R⁶¹, —NR⁶²C(NR⁶³)NR⁶⁰R⁶¹ and —C(NR⁶²)NR⁶⁰R⁶¹ where M is independently a halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. Preferably, substituents include -M, —R⁶⁰, =OR⁶⁰, —SR⁶⁰, —S⁻, ═S, NR⁶⁰R⁶¹, ═NR⁶⁰, —CF₃, —CN, —OCN, —SCN, —NO, —NO₂, ═N₂, —N₃, —S(O)₂R⁶¹, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶¹, —C(O)NR OR⁶⁰, —C(O)O⁻, —NR⁶²C(O)NR⁶⁰R⁶¹, more preferably, -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O—), —OP(O)(OR⁶⁰)(OR⁶¹), —C(O)R⁶⁰, —C(O)OR⁶⁰, —C(O)NR⁶⁰R⁶¹, —C(O)O—, most preferably, -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above.

“Sustained release” refers to release of an agent from a dosage form at a rate effective to achieve a therapeutic or prophylactic amount of the agent, or active metabolite thereof, in the systemic blood circulation over a prolonged period of time relative to that achieved by oral administration of a conventional formulation of the agent. In one embodiment, release of the agent occurs over a period of at least 6 hours. In another embodiment, release of the agent occurs over a period of at least 8 hours. In still another embodiment, release of the agent occurs over a period of at least 12 hours.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter) or both. In yet another embodiment, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### 4.2 Prodrugs of GABA Analogs

In one embodiment, a prodrug of a GABA analog has the structure of Formula (I):

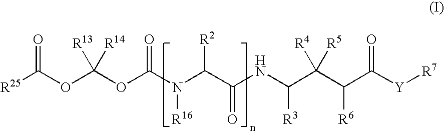

or a pharmaceutically acceptable salt, hydrate, solvate or N-oxide thereof, wherein:

n is 0 or 1;

Y is O or S;

R¹⁶ is hydrogen, alkyl or substituted alkyl;

R² is selected from the group consisting of hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, or optionally, R² and R¹⁶ together with the atoms to which they are attached form a cycloheteroalkyl or substituted cycloheteroalkyl ring;

R³ and R⁶ are independently selected from the group consisting of hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl and substituted heteroarylalkyl;

R⁴ and R⁵ are independently selected from the group consisting of hydrogen, alkyl, substituted alkyl, acyl, substituted acyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl and substituted heteroarylalkyl or optionally, R⁴ and R⁵ together with the carbon atom to which they are attached form a cycloalkyl, substituted cycloalkyl, cycloheteroalkyl, substituted cycloheteroalkyl or bridged cycloalkyl ring;

R⁷ is selected from the group consisting of hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl and substituted heteroarylalkyl;

R¹³ and R¹⁴ are each independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl, substituted heteroaryl, heteroarylalkyl or substituted heteroarylalkyl or optionally, R¹³ and R¹⁴ together with the carbon atom to which they are attached form a cycloalkyl, substituted cycloalkyl, cycloheteroalkyl or substituted cycloheteroalkyl ring; and

R²⁵ is selected from the group consisting of acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl and substituted heteroarylalkyl.

In one embodiment, R¹³ and R¹⁴ are independently hydrogen, alkyl, substituted alkyl, alkoxycarbonyl, aryl, arylalkyl, carbamoyl, cycloalkyl, substituted cycloalkyl or heteroaryl (preferably, when R¹³ is alkoxycarbonyl or carbamoyl then R¹⁴ is methyl). In another embodiment, R¹³ and R¹⁴ are independently hydrogen, methyl, ethyl, propyl, isopropyl, butyl, isobutyl, sec-butyl, tert-butyl, cyclopentyl, cyclohexyl, methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, cyclohexyloxycarbonyl, phenyl, benzyl, phenethyl or 3-pyridyl.

In still another embodiment, R¹³ and R¹⁴ are independently hydrogen, alkanyl, substituted alkanyl, cycloalkanyl or substituted cycloalkanyl. In still another embodiment, R¹³ and R¹⁴ are hydrogen, alkanyl or cycloalkanyl. In still another embodiment, R¹³ and R¹⁴ are independently hydrogen, methyl, ethyl, propyl, isopropyl, butyl, isobutyl, sec-butyl, tert-butyl, cyclopentyl or cyclohexyl. In still another embodiment, R¹³ is methyl, ethyl, propyl, isopropyl, butyl, isobutyl, sec-butyl, tert-butyl, cyclopentyl or cyclohexyl and R¹⁴ is hydrogen, or R¹³ is methyl and R¹⁴ is methyl.

In still another embodiment, R¹³ and R¹⁴ are independently hydrogen, aryl, arylalkyl or heteroaryl. In still another embodiment, R¹³ and R¹⁴ are independently hydrogen, phenyl, benzyl, phenethyl or 3-pyridyl. In still another embodiment, R¹³ is phenyl, benzyl, phenethyl or 3-pyridyl and R¹⁴ is hydrogen.

In still another embodiment, R¹³ and R¹⁴ are independently hydrogen, alkyl, substituted alkyl, alkoxycarbonyl or carbamoyl. In still another embodiment, R¹³ is alkoxycarbonyl or carbamoyl and R¹⁴ is methyl. In still another embodiment, R¹³ is methoxycarbonyl, ethoxycarbonyl, isopropoxycarbonyl, butoxycarbonyl, isobutoxycarbonyl, sec-butoxycarbonyl, tert-butoxycarbonyl or cyclohexyloxycarbonyl and R¹⁴ is methyl.

In still another embodiment, R¹³ and R¹⁴ together with the carbon atom to which they are attached form a cycloalkyl, substituted cycloalkyl, cycloheteroalkyl or substituted cycloheteroalkyl ring. In still another embodiment, R¹³ and R¹⁴ together with the carbon atom to which they are attached form a cycloalkyl ring. In still another embodiment, R¹³ and R¹⁴ together with the carbon atom to which they are attached form a cyclobutyl, cyclopentyl or cyclohexyl ring.

In still another embodiment of compounds of Formula (I), R²⁵ is acyl, substituted acyl, alkyl, substituted alkyl, aryl, arylalkyl, cycloalkyl or heteroaryl. In still another embodiment, R²⁵ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl or 3-pyridyl.

In still another embodiment, R²⁵ is acyl or substituted acyl. In still another embodiment, R²⁵ is acetyl, propionyl, butyryl, benzoyl or phenacetyl.

In still another embodiment, R²⁵ is alkanyl or substituted alkanyl. In still another embodiment, R²⁵ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl or 1-(1,3-dioxan-2-yl)-2-phenethyl. In still another embodiment, R²⁵ is methyl, ethyl, propyl, isopropyl, butyl, 1,1-dimethoxyethyl or 1,1-diethoxyethyl.

In still another embodiment, R²⁵ is aryl, arylalkyl or heteroaryl. In still another embodiment, R²⁵ is phenyl, 4-methoxyphenyl, benzyl, phenethyl, styryl or 3-pyridyl.

In still another embodiment, R²⁵ is cycloalkyl or substituted cycloalkyl. In still another embodiment, R²⁵ is cyclopropyl, cyclobutyl, cyclopentyl or cyclohexyl.

In still another embodiment, R²⁵ is acyl, substituted acyl, alkyl, substituted alkyl, aryl, arylalkyl, cycloalkyl or heteroaryl and R¹³ and R¹⁴ are independently hydrogen, alkyl, substituted alkyl, alkoxycarbonyl, aryl, arylalkyl, carbamoyl, cycloalkyl, or heteroaryl (preferably, R¹³ is alkoxycarbonyl or carbamoyl and R¹⁴ is methyl). In still another embodiment, R²⁵ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1, 1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1, 1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl or 3-pyridyl and R¹³ and R¹⁴ are independently hydrogen, methyl, ethyl, propyl, isopropyl, butyl, isobutyl, sec-butyl, tert-butyl, cyclopentyl, cyclohexyl, methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, cyclohexyloxycarbonyl, phenyl, benzyl, phenethyl or 3-pyridyl. In still another embodiment, R²⁵ is methyl, ethyl, propyl, isopropyl, butyl, isobutyl, sec-butyl, 1,1-dimethoxyethyl, 1,1-diethoxyethyl, 1,1-dimethoxybenzyl, 1,1-diethoxybenzyl, 1,1-dimethoxy-2-phenethyl, 1,1-diethoxy-2-phenethyl, acetyl, propionyl, butyryl, benzoyl, phenacetyl, phenyl, 4-methoxyphenyl, benzyl, phenethyl, cyclohexyl or 3-pyridyl and R¹³ and R¹⁴ are independently hydrogen, methyl, ethyl, propyl, isopropyl, butyl, isobutyl, sec-butyl, tert-butyl, cyclopentyl, cyclohexyl, methoxycarbonyl, ethoxycarbonyl, isopropoxycarbonyl, cyclohexyloxycarbonyl, phenyl, benzyl, phenethyl or 3-pyridyl.

In still another embodiment, R²⁵ is acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl and R¹³ and R¹⁴ together with the atom to which they are attached form a cycloalkyl, substituted cycloalkyl, cycloheteroalkyl or substituted cycloheteroalkyl ring. In still another embodiment, R²⁵ is acyl, substituted acyl, alkyl, substituted alkyl, aryl, arylalkyl, cycloalkyl or heteroaryl and R¹³ and R¹⁴ together with the atom to which they are attached form a cycloalkyl or substituted cycloalkyl ring. In still another embodiment, R²⁵ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1, 1-dimethoxybenzyl, 1,1-diethoxybenzyl, 1-(1,3-dioxolan-2-yl)-benzyl, 1-(1,3-dioxan-2-yl)-benzyl, 1, 1-dimethoxy-2-phenethyl, 1, 1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl or 3-pyridyl and R¹³ and R¹⁴ together with the atom to which they are attached form a cyclobutyl, cyclopentyl or a cyclohexyl ring.

In still another embodiment, R²⁵ is acyl or substituted acyl and R¹³ and R¹⁴ are independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl or substituted heteroaryl (preferably, when R¹³ is alkoxycarbonyl, substituted alkoxycarbonyl, carbamoyl or substituted carbamoyl then R¹⁴ is methyl). In still another embodiment, R²⁵ is acetyl, propionyl, butyryl, benzoyl or phenacetyl, and R¹³ and R¹⁴ are independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl or substituted heteroaryl (preferably, when R¹³ is alkoxycarbonyl, or carbamoyl then R¹⁴ is methyl).

In still another embodiment, R²⁵ is alkanyl or substituted alkanyl and R¹³ and R¹⁴ are independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl or substituted heteroaryl (preferably, when R¹³ is alkoxycarbonyl, substituted alkoxycarbonyl, carbamoyl or substituted carbamoyl then R¹⁴ is methyl). In still another embodiment, R²⁵ is methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl or 1-(1,3-dioxan-2-yl)-2-phenethyl and R¹³ and R¹⁴ are independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl or substituted heteroaryl (preferably, when R¹³ is alkoxycarbonyl or carbamoyl then R¹⁴ is methyl).

In still another embodiment, R²⁵ is aryl, substituted aryl, arylalkyl, substituted arylalkyl, heteroaryl or substituted heteroaryl and R¹³ and R¹⁴ are independently hydrogen, alkyl, substituted alkyl, alkoxycarbonyl, substituted alkoxycarbonyl, aryl, substituted aryl, arylalkyl, substituted arylalkyl, carbamoyl, cycloalkyl, substituted cycloalkyl, heteroaryl or substituted heteroaryl (preferably, when R¹³ is alkoxycarbonyl, substituted alkoxycarbonyl, carbamoyl or substituted carbamoyl then R¹⁴ is methyl). In still another embodiment, R²⁵ is phenyl, 4-methoxyphenyl, benzyl, phenethyl, styryl or 3-pyridyl and R¹³ and R¹⁴ are independently hydrogen, alkyl, substituted alkyl, alkoxycarbonyl, substituted alkoxycarbonyl, aryl, substituted aryl, arylalkyl, substituted arylalkyl, carbamoyl, cycloalkyl, substituted cycloalkyl, heteroaryl or substituted heteroaryl (preferably, when R¹³ is alkoxycarbonyl or carbamoyl then R¹⁴ is methyl).

In still another embodiment, R²⁵ is cycloalkyl or substituted cycloalkyl, and R¹³ and R¹⁴ are independently hydrogen, alkyl, substituted alkyl, alkoxycarbonyl, substituted alkoxycarbonyl, aryl, substituted aryl, arylalkyl, substituted arylalkyl, carbamoyl, cycloalkyl, substituted cycloalkyl, heteroaryl or substituted heteroaryl (preferably, when R¹³ is alkoxycarbonyl, substituted alkoxycarbonyl, carbamoyl or substituted carbamoyl then R¹⁴ is methyl). Preferably, R²⁵ is cyclopropyl, cyclobutyl, cyclopentyl or cyclohexyl and R¹³ and R¹⁴ are independently hydrogen, alkyl, substituted alkyl, alkoxycarbonyl, substituted alkoxycarbonyl, aryl, substituted aryl, arylalkyl, substituted arylalkyl, carbamoyl, cycloalkyl, substituted cycloalkyl, heteroaryl or substituted heteroaryl (preferably, when R¹³ is alkoxycarbonyl, or carbamoyl then R¹⁴ is methyl).

In still another embodiment, R²⁵ is acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl, and R¹³ and R¹⁴ are independently hydrogen, alkyl, substituted alkyl, aryl, arylalkyl, cycloalkyl or heteroaryl. In still another embodiment, R²⁵ is acyl, substituted acyl, alkyl, substituted alkyl, aryl, arylalkyl, cycloalkyl or heteroaryl and R¹³ and R¹⁴ are independently hydrogen, alkanyl, substituted alkanyl, cycloalkanyl or substituted cycloalkanyl. In still another embodiment, R²⁵ is acyl, substituted acyl, alkyl, substituted alkyl, aryl, arylalkyl, cycloalkyl or heteroaryl and R¹³ and R¹⁴ are independently hydrogen, methyl, ethyl, propyl, isopropyl, butyl, isobutyl, sec-butyl, tert-butyl, cyclopentyl or cyclohexyl. In the above embodiments, R²⁵ is preferably methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1, 1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1, 1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl or 3-pyridyl.

In still another embodiment, R²⁵ is acyl, substituted acyl, alkyl, substituted alkyl, aryl, arylalkyl, cycloalkyl or heteroaryl and R¹³ and R¹⁴ are independently hydrogen, alkyl, substituted alkyl, aryl, arylalkyl, cycloalkyl or heteroaryl. In still another embodiment, R²⁵ is acyl, substituted acyl, alkyl, substituted alkyl, aryl, arylalkyl, cycloalkyl or heteroaryl and R¹³ and R¹⁴ are independently hydrogen, aryl, arylalkyl or heteroaryl. In still another embodiment, R²⁵ is acyl, substituted acyl, alkyl, substituted alkyl, aryl, arylalkyl, cycloalkyl or heteroaryl and R¹³ and R¹⁴ are independently hydrogen, phenyl, benzyl, phenethyl or 3-pyridyl. In still another embodiment, R²⁵ is preferably methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl or 3-pyridyl.

In still another embodiment, R²⁵ is acyl, substituted acyl, alkyl, substituted alkyl, aryl, arylalkyl, cycloalkyl or heteroaryl and R¹³ and R¹⁴ are independently hydrogen, alkyl, substituted alkyl, aryl, arylalkyl, cycloalkyl or heteroaryl. In still another embodiment, R²⁵ is acyl, substituted acyl, alkyl, substituted alkyl, aryl, arylalkyl, cycloalkyl or heteroaryl and R¹³ and R¹⁴ are independently hydrogen, alkyl, substituted alkyl, alkoxycarbonyl, substituted alkoxycarbonyl, carbamoyl or substituted carbamoyl, (preferably, when R¹³ is alkoxycarbonyl, substituted alkoxycarbonyl, carbamoyl or substituted carbamoyl then R¹⁴ is methyl, more preferably, R¹³ is methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl or cyclohexyloxycarbonyl, and R¹⁴ is methyl). In the above embodiments, R²⁵ is preferably methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl or 3-pyridyl.

In still another embodiment, R²⁵ is acyl, substituted acyl, alkyl, substituted alkyl, aryl, arylalkyl, cycloalkyl or heteroaryl and R¹³ and R¹⁴ together with the atom to which they are attached form a cycloalkyl, substituted cycloalkyl, cycloheteroalkyl or substituted cycloheteroalkyl ring. In still another embodiment, R²⁵ is acyl, substituted acyl, alkyl, substituted alkyl, aryl, arylalkyl, cycloalkyl or heteroaryl and R¹³ and R¹⁴ together with the atom to which they are attached form a cycloalkyl or substituted cycloalkyl ring. In still another embodiment, R²⁵ is acyl, substituted acyl, alkyl, substituted alkyl, aryl, arylalkyl, cycloalkyl or heteroaryl, and R¹³ and R¹⁴ together with the atom to which they are attached form a cyclobutyl, cyclopentyl or cyclohexyl ring. In still another embodiment, R 25 is preferably methyl, ethyl, propyl, isopropyl, butyl, isobutyl, sec-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl or 3-pyridyl.

In still another embodiment of compounds of Formula (I), R⁴ and R⁵ together with the carbon atom to which they are attached form a cyclobutyl or substituted cyclobutyl ring. In still another embodiment, the substituted cyclobutyl ring is substituted with one or more substituents selected from the group consisting of alkanyl, substituted alkanyl, halo, hydroxy, carboxy and alkoxycarbonyl.

In still another embodiment of compounds of Formula (I), R⁴ and R⁵ together with the carbon atom to which they are attached form a cyclopentyl or substituted cyclopentyl ring In still another embodiment, the cyclopentyl ring is substituted with alkanyl, substituted alkanyl, halo, hydroxy, carboxy or alkoxycarbonyl. In still another embodiment, the cyclopentyl ring is substituted with alkanyl. In still another embodiment, the cyclopentyl ring is selected from the group consisting of

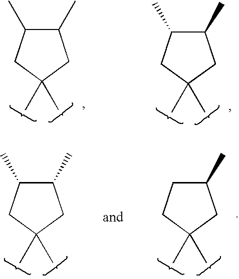

In a more specific version of the above embodiments, R⁷ is hydrogen.

In still another embodiment of compounds of Formula (I), R⁴ and R⁵ together with the carbon atom to which they are attached form a cyclohexyl or substituted cyclohexyl ring. In still another embodiment, the cyclohexyl ring is substituted with alkanyl, substituted alkanyl, halo, hydroxy, carboxy or alkoxycarbonyl. In still another embodiment, the cyclohexyl ring is substituted with alkanyl. In still another embodiment, the cyclohexyl ring is selected from the group consisting of

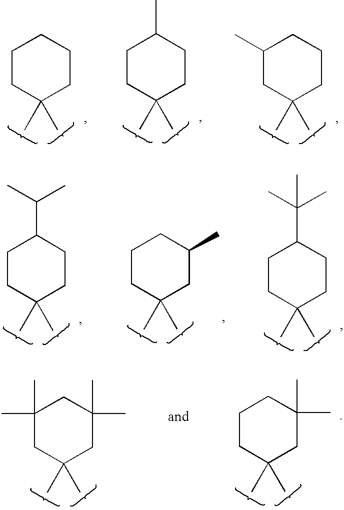

In a more specific version of the above embodiments, R⁷ is hydrogen.

In still another embodiment of compounds of Formula (I), R⁴ and R⁵ together with the carbon atom to which they are attached form a cycloheteroalkyl or substituted cycloheteroalkyl ring. In one embodiment, n is 0. In another embodiment, n is 1, and R² is hydrogen, methyl, 2-propyl, 2-butyl, isobutyl, t-butyl, cyclopentyl, cyclohexyl, phenyl, benzyl, 4-hydroxybenzyl, 4-imidazolylmethyl, 3-indolylmethyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂ or —CH₂CH₂CH₂NHC(NH)NH₂. In still another embodiment, n is 1 and R² and R¹⁶ together with the atoms to which they are attached form a pyrrolidine ring. Preferably, R⁴ and R⁵ together with the carbon atom to which they are attached form a cycloheteroalkanyl ring. More preferably, the cycloheteroalkanyl ring is selected from the group consisting of

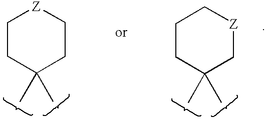

wherein Z is O, S(O)ₚ or NR¹⁸;

p is 0, 1 or 2; and

R¹⁸ is selected from the group consisting of hydrogen, alkyl, substituted alkyl, acyl and alkoxycarbonyl. More preferably, the cycloheteroalkanyl ring is selected from the group consisting of

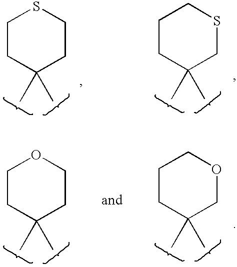

In a more specific version of the above embodiments, R⁷ is hydrogen.

In still another embodiment of compounds of Formula (I), R⁴ and R⁵ together with the carbon atom to which they are attached form a bridged cycloalkyl ring. In one embodiment, n is 0. In another embodiment, n is 1 and R² is hydrogen, methyl, 2-propyl, 2-butyl, isobutyl, t-butyl, cyclopentyl, cyclohexyl, phenyl, benzyl, 4-hydroxybenzyl, 4-imidazolylmethyl, 3-indolylmethyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂ or —CH₂CH₂CH₂NHC(NH)NH₂. In another embodiment, n is 1 and R and R together with the atoms to which they are attached form a pyrrolidine ring. Preferably, the bridged cycloalkyl group is

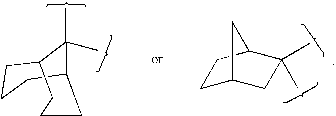

In a more specific version of the above embodiments, R⁷ is hydrogen.

In still another embodiment of compounds of Formula (I), Y is 0, R⁶ and R⁷ are hydrogen, R⁴ is alkyl or cycloalkyl, R⁵ is hydrogen or alkyl and R³ is hydrogen or alkyl. In one embodiment, n is 0. In another embodiment, n is 1 and R² is hydrogen, methyl, 2-propyl, 2-butyl, isobutyl, t-butyl, cyclopentyl, cyclohexyl, phenyl, benzyl, 4-hydroxybenzyl, 4-imidazolylmethyl, 3-indolylmethyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂ or —CH₂CH₂CH₂NHC(NH)NH₂. In another embodiment, n is 1 and R² and R¹⁶ together with the atoms to which they are attached form a pyrrolidine ring. Preferably, R⁴ is cycloalkyl, R⁵ is hydrogen or methyl, and R³ is hydrogen or methyl. Preferably, R³ is hydrogen, R⁴ is isobutyl and R⁵ is hydrogen.

In still another embodiment of compounds of Formula (I), Y is 0, R⁵ and R⁷ are hydrogen or alkanyl, R³ and R⁶ are hydrogen and R⁴ is substituted heteroalkyl. Preferably, R⁴ is

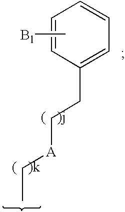

A is NR¹⁹, O or S;

B is alkyl, substituted alkyl, alkoxy, halogen, hydroxy, carboxy, alkoxycarbonyl or amino;

R¹⁹ is hydrogen, alkyl, cycloalkyl or aryl;

j is an integer from 0 to 4;

k is an integer from 1 to 4; and

l is an integer from 0 to 3.

More preferably, k is 1.

In still another embodiment of compounds of Formula (I), Y is 0, R⁵ and R⁷ are hydrogen or alkanyl, R³ and R⁶ are hydrogen and R⁴ is substituted alkanyl, cycloalkanyl or substituted cycloalkanyl. Preferably, R⁴ is selected from the group consisting of

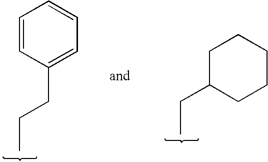

Preferably, R⁴ is

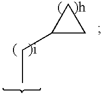

h is an integer from 1 to 6; and

i is an integer from 0 to 6.

More preferably, h is 1, 2, 3 or 4 and i is 0 or 1. Even more preferably, R⁴ is selected from the group consisting of

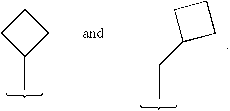

Preferably, compounds of Formula (I) are derived from a GABA analog of Formula (IV):

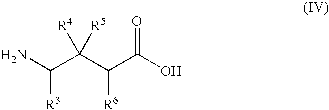

wherein the GABA analog of Formula (IV) is selected from the group consisting of 1-Aminomethyl-1-cyclohexane acetic acid (i.e., gabapentin), 1-Aminomethyl-1-(3-methylcyclohexane) acetic acid; 1-Aminomethyl-1-(4-methylcyclohexane) acetic acid, 1-Aminomethyl-1-(4-isopropylcyclohexane) acetic acid, 1-Aminomethyl-1-(4-tert-butylcyclohexane) acetic acid, 1-Aminomethyl-1-(3,3-dimethylcyclohexane) acetic acid, 1-Aminomethyl-1-(3,3,5,5-tetramethylcyclohexane) acetic acid, 1-Aminomethyl-1-cyclopentane acetic acid, 1-Aminomethyl-1-(3-methylcyclopentane) acetic acid, 1-Aminomethyl-1-(3,4-dimethylcyclopentane) acetic acid, 7-Aminomethyl-bicyclo[2.2.1]hept-7-yl acetic acid; 9-Aminomethyl-bicyclo[3.3.1]non-9-yl acetic acid, 4-Aminomethyl-4-(tetrahydropyran-4-yl) acetic acid, 3-Aminomethyl-3-(tetrahydropyran-3-yl) acetic acid, 4-Aminomethyl-4-(tetrahydrothiopyran-4-yl) acetic acid, 3-Aminomethyl-3-(tetrahydrothiopyran-3-yl) acetic acid, (S)-3-Aminomethyl-5-methyl-hexanoic acid (i.e., pregabalin), 3-Aminomethyl-5-methyl-heptanoic acid, 3-Aminomethyl-5-methyl-octanoic acid, 3-Aminomethyl-5-methyl-nonanoic acid, 3-Aminomethyl-5-methyl-decanoic acid, 3-Aminomethyl-5-cyclopropyl-hexanoic acid, 3-Aminomethyl-5-cyclobutyl-hexanoic acid, 3-Aminomethyl-5-cyclopentyl-hexanoic acid, 3-Aminomethyl-5-cyclohexyl-hexanoic acid, 3-Aminomethyl-5-phenyl-hexanoic acid, 3-Aminomethyl-5-phenyl-pentanoic acid, 3-Aminomethyl-4-cyclobutyl-butyric acid, 3-Aminomethyl-4-cyclopentyl-butyric acid, 3-Aminomethyl-4-cyclohexyl-butyric acid, 3-Aminomethyl-4-phenoxy-butyric acid, 3-Aminomethyl-5-phenoxy-hexanoic acid and 3-Aminomethyl-5-benzylsulfanyl-pentanoic acid.

In still another embodiment, compounds of Formula (I) have the structure of Formulae (II) and (III):

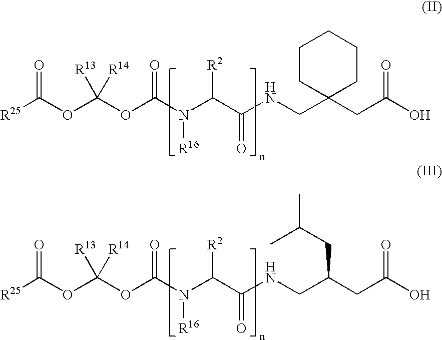

wherein n, R², R¹³, R¹⁴, R¹⁶ and R²⁵ are as previously defined.

In one embodiment of compounds of Formulae (II) and (III), n is 0. In another embodiment, n is 1. When n is 1, preferably, the α-amino acid is of the L-stereochemical configuration.

In still another embodiment of compounds of Formulae (II) and (III), n is 1, R¹⁶ is hydrogen and R² is hydrogen, methyl, 2-propyl, 2-butyl, isobutyl, tert-butyl, cyclopentyl, cyclohexyl, phenyl, benzyl, 4-hydroxybenzyl, 4-imidazolylmethyl, 3-indolylmethyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂ or —CH₂CH₂CH₂NHC(NH)NH₂. In still another embodiment, R¹⁶ is hydrogen and R² is hydrogen, methyl, 2-propyl, 2-butyl, isobutyl, tert-butyl, cyclohexyl, phenyl or benzyl. In still another embodiment, n is 1 and R² and R¹⁶ together with the atoms to which they are attached form a pyrrolidine ring.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1, 1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is hydrogen and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1, 1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is methyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is ethyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is propyl and R¹⁴ is hydrogen.

In still another embodiment of compounds of Formulae (H) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is isopropyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is butyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1, 1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is isobutyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1, 1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is sec-butyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is tert-butyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1, 1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is cyclopentyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is cyclohexyl and R¹⁴ is hydrogen.

In still another embodiment of compounds of Formulae (11) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is methyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is methoxycarbonyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is ethoxycarbonyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is propoxycarbonyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1, 1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1, 1-diethoxybenzyl, 1-(1,3-dioxolan-2-yl)-benzyl, 1-(1,3-dioxan-2-yl)-benzyl, 1,1-dimethoxy-2-phenethyl, 1, 1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is isopropoxycarbonyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is butoxycarbonyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is isobutoxycarbonyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1, 1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is sec-butoxycarbonyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is tert-butoxycarbonyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is cyclohexyloxycarbonyl and R¹⁴ is methyl.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1, 1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is phenyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is benzyl and R¹⁴ is hydrogen.

In still another embodiment of compounds of Formulae (II) and (III), R²⁵ is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is phenethyl and R¹⁴ is hydrogen.

In still another embodiment of compounds of Formulae (II) and (III), R25 is selected from the group consisting of methyl, ethyl, propyl, isopropyl, butyl, isobutyl, sec-butyl, tert-butyl, pentyl, isopentyl, sec-pentyl, neopentyl, 1,1-dimethoxyethyl, 1,1-diethoxyethyl, 1-(1,3-dioxolan-2-yl)-ethyl, 1-(1,3-dioxan-2-yl)-ethyl, 1,1-dimethoxypropyl, 1,1-diethoxypropyl, 1-(1,3-dioxolan-2-yl)-propyl, 1-(1,3-dioxan-2-yl)-propyl, 1,1-dimethoxybutyl, 1,1-diethoxybutyl, 1-(1,3-dioxolan-2-yl)-butyl, 1-(1,3-dioxan-2-yl)-butyl, 1,1-dimethoxybenzyl, 1,1-diethoxybenzyl, 1-(1,3-dioxolan-2-yl)-benzyl, 1-(1,3-dioxan-2-yl)-benzyl, 1,1-dimethoxy-2-phenethyl, 1,1-diethoxy-2-phenethyl, 1-(1,3-dioxolan-2-yl)-2-phenethyl, 1-(1,3-dioxan-2-yl)-2-phenethyl, acetyl, propionyl, butyryl, benzoyl, phenacetyl, phenyl, 4-methoxyphenyl, benzyl, phenethyl, styryl, cyclopropyl, cyclobutyl, cyclopentyl, cyclohexyl and 3-pyridyl, R¹³ is 3-pyridyl and R¹⁴ is hydrogen.

In still another embodiment, compounds of Formulae (II) and (III) have the structure of Formulae (V) and (VI), respectively

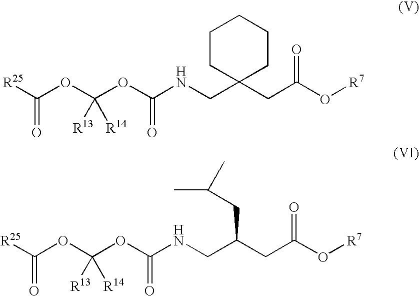

or a pharmaceutically acceptable salt, hydrate, solvate or N-oxide thereof where R⁷ and R¹⁴ are each hydrogen, R¹³ is C₁-C₆ and R²⁵ is C₁-C₆ alkyl or C₁-C₆ substituted alkyl. Preferably, R¹³ is selected from the group consisting of methyl, ethyl, n-propyl, isopropyl, n-butyl, isobutyl and sec-butyl and R²⁵ is selected from the group consisting of methyl, ethyl, n-propyl, isopropyl, n-butyl, isobutyl, sec-butyl, n-pentyl, isopentyl, sec-pentyl, neopentyl and 1,1-diethoxyethyl.

In one embodiment of compounds of Formulae (V) and (VI), R¹³ is methyl. In another embodiment of compound of compounds of Formulae (V) and (VI), R²⁵ is methyl, ethyl, n-propyl or isopropyl. In still another embodiment of compounds of Formulae (V) and (VI), R¹³ is methyl and R²⁵ is methyl, ethyl, n-propyl or n-butyl. In still another embodiment of compounds of Formulae (V) and (VI), R¹³ is ethyl and R²⁵ is methyl, n-propyl or isopropyl. In still another embodiment of compounds of Formulae (V) and (VI), R¹³ is n-propyl and R²⁵ is methyl, ethyl, n-propyl, isopropyl or n-butyl. In still another embodiment of compounds of Formulae (V) and (VI), R¹³ is isopropyl and R²⁵ is methyl, ethyl, n-propyl, isopropyl, n-butyl or isobutyl. In still another embodiment of compounds of Formulae (V) and (VI), R¹³ is n-propyl and R²⁵ is n-propyl. In still another embodiment of compounds of Formulae (V) and (VI), R¹³ is methyl and R²⁵ is ethyl. In still another embodiment of compounds of Formulae (V) and (VI), R is methyl and R²⁵ is isopropyl. In still another embodiment of compounds of Formulae (V) and (VI), R¹³ is isopropyl and R²⁵ is isopropyl. In still another embodiment of compounds of Formulae (V) and (VI), R¹³ is isopropyl and R²⁵ is 1,1-diethoxyethyl. In still another embodiment of compounds of Formulae (V) and (VI), R¹³ is propyl and R²⁵ is isopropyl. In still another embodiment of compounds of Formulae (V) and (VI), R¹³ is propyl and R²⁵ is ethyl.

In one embodiment, the compound of Formula (V) where R²⁵ is isopropyl, R¹³ is methyl and R¹⁴ is hydrogen is a crystalline form of 1-{[(α-isobutanoyloxyethoxy)carbonyl]aminomethyl}-1-cyclohexane acetic acid as disclosed in Estrada et al., U.S. patent application Ser. No. ______, which claims the benefit of U.S. Provisional Application Ser. No. 60/511,287, filed Oct. 14, 2003.

Specific examples of Formula (V) compounds include 1-{[(α-acetoxyethoxy)carbonyl]aminomethyl}-1-cyclohexane acetic acid, 1-{[(α-propanoyloxyethoxy)carbonyl]aminomethyl}-1-cyclohexane acetic acid, 1-{[(α-butanoyloxyethoxy)carbonyl]aminomethyl}-1-cyclohexane acetic acid, 1-{[(α-isobutanoyloxyethoxy)carbonyl]aminomethyl}-1-cyclohexane acetic acid, 1-{[(α-pivaloxyethoxy)carbonyl]aminomethyl}-1-cyclohexane acetic acid, 1-{[(α-acetoxymethoxy)carbonyl]aminomethyl}-1-cyclohexane acetic acid, 1-{[(α-propanoyloxymethoxy)carbonyl]aminomethyl}-1-cyclohexane acetic acid, 1-{[(α-butanoyloxymethoxy)carbonyl]aminomethyl}-1-cyclohexane acetic acid, 1-{[(α-isobutanoyloxymethoxy)carbonyl]aminomethyl}-1-cyclohexane acetic acid, 1-{[(α-pivaloxymethoxy)carbonyl]aminomethyl}-1-cyclohexane acetic acid, 1-{[(α-acetoxypropoxy)carbonyl]aminomethyl}-1-cyclohexane acetic acid, 1-{[(α-propanoyloxypropoxy)carbonyl]aminomethyl}-1-cyclohexane acetic acid, 1-{[(α-butanoyloxypropoxy)carbonyl]aminomethyl}-1-cyclohexane acetic acid, 1-{[(α-isobutanoyloxypropoxy)carbonyl]aminomethyl}-1-cyclohexane acetic acid, 1-{[(α-pivaloxypropoxy)carbonyl]aminomethyl}-1-cyclohexane acetic acid, 1-{[(α-acetoxyisopropoxy)carbonyl]aminomethyl}-1-cyclobexane acetic acid, 1-{[(α-propanoyloxyisopropoxy)carbonyl]aminomethyl}-1-cyclohexane acetic acid, 1-{[(α-butanoyloxyisopropoxy)carbonyl]aminomethyl}-1-cyclohexane acetic acid, 1-{[(α-isobutanoyloxyisopropoxy)carbonyl]aminomethyl}-1-cyclohexane acetic acid, 1-{[(α-pivaloxyisopropoxy)carbonyl]aminomethyl}-1-cyclohexane acetic acid, 1-{[(αacetoxybutoxy)carbonyl]aminomethyl}-1-cyclohexane acetic acid, 1-{[(α--propanoyloxybutoxy)carbonyl]aminomethyl}-1-cyclohexane acetic acid 1-{[(α-butanoyloxybutoxy)carbonyl]aminomethyl}-1-cyclohexane acetic acid 1-{[(α-isobutanoyloxybutoxy)carbonyl]aminomethyl}-1-cyclohexane acetic acid and 1-{[(α-pivaloxybutoxy)carbonyl]aminomethyl}-1-cyclohexane acetic acid.

Specific examples of Formula (VI) compounds include 3-{[(α-acetoxyethoxy)carbonyl]aminomethyl}-5-methyl hexanoic acid, 3-{[(α-propanoyloxyethoxy)carbonyl]aminomethyl}-5-methyl hexanoic acid, 3-{[(═-butanoyloxyethoxy)carbonyl]aminomethyl}-5-methyl hexanoic acid, 3-{[(α-isobutanoyloxyethoxy)carbonyl]aminomethyl}-5-methyl hexanoic acid, 3-{[(α-pivaloxyethoxy)carbonyl]aminomethyl}-5-methyl hexanoic acid, 3-{[(α-acetoxymethoxy)carbonyl]aminomethyl}-5-methyl hexanoic acid, 3-{[(α-propanoyloxymethoxy)carbonyl]aminomethyl}-5-methyl hexanoic acid, 3-{[(α-butanoyloxymethoxy)carbonyl]aminomethyl}-5-methyl hexanoic acid, 3-{[(α-isobutanoyloxymethoxy)carbonyl]aminomethyl}-5-methyl hexanoic acid, 3-{[(α-pivaloxymethoxy)carbonyl]aminomethyl}-5-methyl hexanoic acid, 3-{[(α-acetoxypropoxy)carbonyl]aminomethyl}-5-methyl hexanoic acid, 3-{[(α-propanoyloxypropoxy)carbonyl]aminomethyl}-5-methyl hexanoic acid, 3-{[(α-butanoyloxypropoxy)carbonyl]aminomethyl}-5-methyl hexanoic acid, 3-{[(α-isobutanoyloxypropoxy)carbonyl]aminomethyl}-5-methyl hexanoic acid, 3-{[(α-pivaloxypropoxy)carbonyl]aminomethyl}-5-methyl hexanoic acid, 3-{[(α-acetoxyisopropoxy)carbonyl]aminomethyl}-5-methyl hexanoic acid, 3-{[(α-propanoyloxyisopropoxy)carbonyl]aminomethyl}-5-methyl hexanoic acid, 3-{[(α-butanoyloxyisopropoxy)carbonyl]aminomethyl}-5-methyl hexanoic acid, 3-{[(α-isobutanoyloxyisopropoxy)carbonyl]aminomethyl}-5-methyl hexanoic acid, 3-{[(α-pivaloxyisopropoxy)carbonyl]aminomethyl}-5-methyl hexanoic acid, 3-{[(α-acetoxybutoxy)carbonyl]aminomethyl}-5-methyl hexanoic acid, 3-{[(α-propanoyloxybutoxy)carbonyl]aminomethyl}-5-methyl hexanoic acid, 3-{[(α-butanoyloxybutoxy)carbonyl]aminomethyl}-5-methyl hexanoic acid, 3-{[(α-isobutanoyloxybutoxy)carbonyl]aminomethyl}-5-methyl hexanoic acid and 3-{[(α-pivaloxybutoxy)carbonyl]aminomethyl}-5-methyl hexanoic acid.

### 4.3 Methods of Synthesis of Prodrugs of GABA Analogs

Methods of synthesis of prodrugs of GABA analogs, including methods of synthesizing compounds of structural Formulae (I), (II), (III), (V) and (VI) are disclosed in Gallop et al., International Publication No. WO 02/100347, Gallop et al., U.S. application Ser. No. 10/313,825, filed Dec. 6, 2002 and Bhat et al., U.S. patent application Ser. No. ______, which claims the benefit of U.S. Provisional Application Ser. No. 60/487,642, filed Jul. 15, 2003. Other methods for synthesis of prodrugs of GABA analogs have also been disclosed (see Bryans et al., International Publication No. WO 01/90052; U.K. Application GB 2,362,646; European Applications EP 1,201,240 and 1,178,034; Yatvin et al., U.S. Pat. No. 6,024,977; Gallop et al., International Publication No. WO 02/28881; Gallop et al., International Publication No. WO 02/28883; Gallop et al., International Publication No. WO 02/28411; Gallop et al., International Publication No. WO 02/32376; Gallop et al., International Publication No. WO 02/42414).

### 4.4 Therapeutic Uses of Prodrugs of GABA Analogs

In one embodiment, a prodrug of a GABA analog and/or pharmaceutical composition thereof is administered to a patient suffering from hot flashes. In another embodiment, a prodrug of a GABA analog and/or pharmaceutical compositions thereof is administered to a patient as a preventative measure against hot flashes. The suitability of GABA analog prodrugs and/or pharmaceutical compositions thereof to treat or prevent hot flashes may be readily determined by methods known to the skilled artisan. The present methods encompass either reducing or preventing the number and/or frequency of hot flashes, reducing or preventing the severity of hot flashes or both.

The patient is a mammal, preferably a human. The patient may be either female or male, although those of skill in the art will appreciate that the cause of hot flashes can be markedly different for either sex. For example, in female patients hot flashes are a primary symptom resulting from menopausal or postmenopausal hormonal variation. However, hot flashes can also be drug-induced by anti-estrogen compounds (e.g., tamoxifen, toremifene, raloxifene, etc.) or surgically-induced by removal of estrogen-producing tissues (e.g., total abdominal hysterectomy, bilateral salpingo-oophorectomy, etc.). In male patients, hot flashes typically occur as a side-effect of androgen-dependent therapy for metastatic prostate cancer. They can be either surgically-induced (e.g., bilateral orchiectomy) or drug-induced, e.g., treatment with gonadotrophin-releasing-hormone analogs (e.g., leuprolide acetate, goserelin acetate, nafarelin acetate, etc.) and anti-androgens (e.g., bicalutamide, flutamide, etc.).

The compounds disclosed herein, particularly the gabapentin prodrug 1-{[(α-isobutanoyloxyethoxy)carbonyl]aminomethyl}-1-cyclohexane acetic acid, may be more efficacious than the parent drug molecule (e.g., gabapentin or other GABA analog) in treating or preventing hot flashes because the disclosed compounds require less time to reach a therapeutic concentration in the blood, i.e., the compounds disclosed herein have a shorter Tₘₐₓ than their parent drug counterparts when taken orally. Without wishing to bound by theory, it is believed that the compounds disclosed herein, particularly the gabapentin prodrug 1-{[(α-isobutanoyloxyethoxy)carbonyl]aminomethyl}-1-cyclohexane acetic acid, are absorbed from the gastrointestinal lumen into the blood by a different mechanism than that by which gabapentin and other known GABA analogs are absorbed. For example, gabapentin is believed to be actively transported across the gut wall by a carrier transporter localized in the human small intestine. The gabapentin transporter is easily saturated which means that the amount of gabapentin absorbed into the blood may not be proportional to the amount of gabapentin that is administered orally, since once the transporter is saturated, further absorption of gabapentin does not occur to any significant degree. In comparison to gabapentin, the compounds disclosed herein, particularly, the gabapentin prodrug 1-{[(α-isobutanoyloxyethoxy)carbonyl]aminomethyl}-1-cyclohexane acetic acid, are absorbed across the gut wall along a greater portion of the gastrointestinal tract, including the colon.

Because the compounds disclosed herein can be formulated in sustained release formulations which provide for sustained release over a period of hours into the gastrointestinal tract and particularly, release within the colon, the compounds (especially, the gabapentin prodrug 1-{[(α-isobutanoyloxyethoxy)carbonyl]aminomethyl}-1-cyclohexane acetic acid) may also be more efficacious than their respective parent drugs (e.g., gabapentin or other GABA analog) in treating or preventing hot flashes. The ability of the compounds disclosed herein to be used in sustained release oral dosage forms may reduce the dosing frequency necessary for maintenance of a therapeutically effective drug concentration in the blood.

### 4.5 Therapeutic/Prophylactic Administration

Dosage forms containing prodrugs of GABA analogs may be advantageously used to treat or prevent hot flashes. The dosage forms may be administered or applied singly, or in combination with other agents. The dosage forms may also deliver a prodrug of a GABA analog to a patient in combination with another pharmaceutically active agent, including another prodrug of a GABA analog. The patient is a mammal and more preferably, a human.

When used in the present methods of treatment, the dosage forms upon releasing a prodrug of a GABA analog in vivo, preferably provide the GABA analog (e.g., gabapentin or pregabalin) in the systemic circulation of the patient. While not wishing to bound by theory, the promoiety or promoieties of the prodrug may be cleaved either chemically and/or enzymatically. One or more enzymes present in the stomach, intestinal lumen, intestinal tissue, blood, liver, brain or any other suitable tissue of a mammal may cleave the promoiety or promoieties of the prodrug. The mechanism of cleavage is not important to the current methods. Preferably, the GABA analog that is formed by cleavage of the promoiety from the prodrug does not contain substantial quantities of lactam contaminant (preferably, less than 0.5% by weight, more preferably, less than 0.2% by weight, most preferably less than 0.1% by weight) for the reasons described in Augart et al., U.S. Pat. No. 6,054,482. The extent of release of lactam contaminant from the prodrugs may be assessed using standard in vitro analytical methods.

Some therapeutically effective GABA analogs, namely gabapentin and pregabalin, have poor passive permeability across the gastrointestinal mucosa (likely due to their zwitterionic characteristics). Although these two GABA analog drugs are actively transported across the gastrointestinal tract by one or more amino acid transporters (e.g. the “large neutral amino acid transporter”), this transporter is expressed predominantly within cells lining the lumen of a limited region of small intestine. This creates a limited window for drug absorption, and an overall dose-dependent drug bioavailability that decreases with increasing dose. A preferred class of GABA analog prodrugs is those that are suitable for oral administration. With such orally administered GABA analog prodrugs, the promoiety or promoieties are preferably cleaved after absorption by the gastrointestinal tract (e.g., in intestinal tissue, blood, liver or other suitable tissue of the patient). In the case of GABA analogs that are poorly absorbed across the gastrointestinal tract mucosa (e.g., gabapentin and pregabalin), the promoiety or promoieties can be designed to make the prodrug a substrate for one or more transporters expressed in the large intestine (i.e., colon), and/or to be passively absorbed across the mucosa.

### 4.6 Pharmaceutical Compositions

The pharmaceutical compositions disclosed herein contain a therapeutically effective amount of one or more GABA analog prodrugs, preferably in purified form, together with a suitable amount of a pharmaceutically acceptable vehicle, so as to provide the form for proper administration to a patient. When administered to a patient, the prodrug and pharmaceutically acceptable vehicles are preferably sterile. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds disclosed herein into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 19th Edition, 1995). Preferred pharmaceutical compositions are formulated for oral delivery, particularly for oral sustained release administration.

Pharmaceutical compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcamitines and the like may be added.

When a GABA analog prodrug is acidic, it may be included in any of the above-described formulations as the free acid, a pharmaceutically acceptable salt, a solvate or hydrate. Pharmaceutically acceptable salts substantially retain the activity of the free acid, may be prepared by reaction with bases, and tend to be more soluble in aqueous and other protic solvents than the corresponding free acid form.

The pharmaceutical compositions preferably contain no or only low levels of lactam side products formed by intramolecular cyclization of the GABA analog and/or GABA analog prodrug. In a preferred embodiment, the pharmaceutical compositions are stable to extended storage (preferably, greater than one year) without substantial lactam formation (preferably, less than 0.5% lactam by weight, more preferably, less than 0.2% lactam by weight, most preferably, less than 0.1% lactam by weight).

### 4.7 Sustained Release Oral Dosage Forms

For those methods that involve oral administration of a GABA analog prodrug to treat or prevent hot flashes, the methods can be practiced with a number of different dosage forms, which provide sustained release of the prodrug upon oral administration. Such sustained release oral dosage forms are particularly preferred for administering those GABA analog prodrugs that are absorbed by cells lining the large intestine, since such dosage forms are generally well adapted to deliver a prodrug to that location of the gastrointestinal tract.

In one embodiment of the invention, the dosage form comprises beads that on dissolution or diffusion release the prodrug over an extended period of hours, preferably, over a period of at least 6 hours, more preferably, over a period of at least 8 hours and most preferably, over a period of at least 12 hours. The prodrug-releasing beads may have comprise a central composition or core comprising a prodrug and pharmaceutically acceptable vehicles, including an optional lubricant, antioxidant and buffer. The beads may be medical preparations with a diameter of about 1 to about 2 mm. Individual beads may comprise doses of the prodrug, for example, doses of up to about 40 mg of prodrug. The beads, in one embodiment, are formed of non-cross-linked materials to enhance their discharge from the gastrointestinal tract. The beads may be coated with a release rate-controlling polymer that gives a timed release profile.

The timed release beads are may be manufactured into a tablet for therapeutically effective prodrug administration. The beads can be are made into matrix tablets by the direct compression of a plurality of beads coated with, for example, an acrylic resin and blended with excipients such as hydroxypropylmethyl cellulose. The manufacture of beads has been disclosed in the art is disclosed in (Lu, Int. J. Pharm. 112, ppl 17-124 (1994); Pharmaceutical Sciences by Remington, 14^{th} ed, pp1626-1628 (1970); Fincher, J. Pharm. Sci. 1968, 57, pp 1825-1835; Benedikt, U.S. Pat. No. 4,083,949) as has the manufacture of tablets (Pharmaceutical Sciences, by Remington, 17^{th} Ed, Ch. 90, pp1603-1625 (1985)).

In another embodiment, an oral sustained release pump may be used (Langer, supra; Sefton, 1987, CRC Crit Ref Biomed. Eng. 14:201; Saudek et al., 1989, N. Engl. J. Med. 321:574).

In another embodiment, polymeric materials can be used (See “Medical Application Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, New York (1984); Langer et al., 1983, J Macromol. Sci. Rev. Macromol Chem. 23:61; Levy et al., 1985, Science 228: 190; During et al., 1989, Ann. Neurol. 25:351; Howard et al., 1989, J. Neurosurg. 71:105). In a preferred embodiment, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropylmethylcellulose). Other preferred cellulose ethers have been described (Alderman, Int. J. Pharm. Tech. & Prod. Mfr. 1984, 5(3) 1-9). Factors affecting drug release are well known to the skilled artisan and have been described in the art (Bamba et al., Int. J. Pharm. 1979, 2, 307).

In another embodiment, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In yet another embodiment, drug-releasing lipid matrices can be used for oral sustained release administration. One particularly preferred example is when solid microparticles of the prodrug are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700. The lipid-coated particles can optionally be compressed to form a tablet. Another controlled release lipid-based matrix material which is suitable for sustained release oral administration comprises polyglycolized glycerides as disclosed in Roussin et al., U.S. Pat. No. 6,171,615.

In yet another embodiment, prodrug-releasing waxes can be used for oral sustained release administration. Examples of suitable sustained prodrug-releasing waxes are disclosed in Cain et al., U.S. Pat. No. 3,402,240 (camauba wax, candedilla wax, esparto wax and ouricury wax); Shtohryn et al., U.S. Pat. No. 4,820,523 (hydrogenated vegetable oil, bees wax, caranuba wax, paraffin, candelillia, ozokerite and mixtures thereof); and Walters, U.S. Pat. No. 4,421,736 (mixture of paraffin and castor wax).

In still another embodiment, osmotic delivery systems are used for oral sustained release administration (Verma et al., Drug Dev. Ind. Pharm. 2000, 26:695-708). In a preferred embodiment, OROS® systems made by Alza Corporation, Mountain View, Calif. are used for oral sustained release delivery devices (Theeuwes et al., U.S. Pat. No. 3,845,770; Theeuwes et al., U.S. Pat. No. 3,916,899).

In yet another embodiment, a controlled-release system can be placed in proximity of the target thus, requiring only a fraction of the systemic dose (Goodson, in “Medical Applications of Controlled Release,” supra, vol. 2, pp. 115-138 (1984)). Other controlled-release systems discussed in Langer, 1990, Science 249:1527-1533 may also be used.

In another embodiment, the dosage form comprises a prodrug of a GABA analog coated on a polymer substrate. The polymer can be an erodible, or a nonerodible polymer. The coated substrate may be folded onto itself to provide a bilayer polymer drug dosage form. For example, a prodrug of a GABA analog can be coated onto a polymer such as a polypeptide, collagen, gelatin, polyvinyl alcohol, polyorthoester, polyacetyl, or a polyorthocarbonate, and the coated polymer folded onto itself to provide a bilaminated dosage form. In operation, the bioerodible dosage form erodes at a controlled rate to dispense the prodrug over a sustained release period. Representative biodegradable polymers comprise a member selected from the group consisting of biodegradable poly(amides), poly (amino acids), poly(esters), poly(lactic acid), poly(glycolic acid), poly(carbohydrate), poly(orthoester), poly (orthocarbonate), poly(acetyl), poly(anhydrides), biodegradable poly(dihydropyrans), and poly(dioxinones) which are known into the art in (Rosoff, Controlled Release of Drugs, Chap. 2, pp. 53-95 (1989); Heller et al., U.S. Pat. No. 3,811,444; Michaels, U.S. Pat. No. 3,962,414; Capozza, U.S. Pat. No. 4,066,747; Schmitt, U.S. Pat. No. 4,070,347; Choi et al., U.S. Pat. No. 4,079,038; Choi et al., U.S. Pat. No. 4,093,709).

In another embodiment, the dosage form comprises a prodrug loaded into a polymer that releases the prodrug by diffusion through a polymer, or by flux through pores or by rupture of a polymer matrix. The drug delivery polymeric dosage form comprises a concentration of 10 mg to 2500 mg homogenously contained in or on a polymer. The dosage form comprises at least one exposed surface at the beginning of dose delivery. The non-exposed surface, when present, is coated with a pharmaceutically acceptable material impermeable to the passage of a prodrug. The dosage form may be manufactured by procedures known in the art. An example of providing a dosage form comprises blending a pharmaceutically acceptable carrier like polyethylene glycol, with a known dose of prodrug at an elevated temperature, (e.g., 37° C.), and adding it to a silastic medical grade elastomer with a cross-linking agent, for example, octanoate, followed by casting in a mold. The step is repeated for each optional successive layer. The system is allowed to set for about 1 hour, to provide the dosage form. Representative polymers for manufacturing the dosage form comprise a member selected from the group consisting of olefin, and vinyl polymers, addition polymers, condensation polymers, carbohydrate polymers, and silicone polymers as represented by polyethylene, polypropylene, polyvinyl acetate, polymethylacrylate, polyisobutylmethacrylate, poly alginate, polyamide and polysilicone. The polymers and procedures for manufacturing them have been described in the art (Coleman et al., Polymers 1990, 31, 1187-1231; Roerdink et al., Drug Carrier Systems 1989, 9, 57-10; Leong et al., Adv. Drug Delivery Rev. 1987, 1, 199-233; Roff et al., Handbook of Common Polymers 1971, CRC Press; Chien et al., U.S. Pat. No. 3,992,518).

In another embodiment, the dosage from comprises a plurality of tiny pills. The tiny time-release pills provide a number of individual doses for providing various time doses for achieving a sustained-release prodrug delivery profile over an extended period of time up to 24 hours. The matrix comprises a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, grum tragacanth, locust bean gum, pectin, amylopectin, gelatin, and a hydrophilic colloid. The hydrophilic matrix comprises a plurality of 4 to 50 tiny pills, each tiny pill comprise a dose population of from 10 ng, 0.5 mg, 1 mg, 1.2 mg, 1.4 mg, 1.6 mg, 5.0 mg etc. The tiny pills comprise a release rate-controlling wall of 0.001 mm up to 10 mm thickness to provide for the timed release of prodrug. Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills are disclosed in Urquhart et al., U.S. Pat. No. 4,434,153; Urquhart et al., U.S. Pat. No. 4,721,613; Theeuwes, U.S. Pat. No. 4,853,229; Barry, U.S. Pat. No. 2,996,431; Neville, U.S. Pat. No. 3,139,383; Mehta, U.S. Pat. No. 4,752,470.

In another embodiment, the dosage form comprises an osmotic dosage form, which comprises a semipermeable wall that surrounds a therapeutic composition comprising the prodrug. In use within a patient, the osmotic dosage form comprising a homogenous composition, imbibes fluid through the semipermeable wall into the dosage form in response to the concentration gradient across the semipermeable wall. The therapeutic composition in the dosage form develops osmotic pressure differential that causes the therapeutic composition to be administered through an exit from the dosage form over a prolonged period of time up to 24 hours (or even in some cases up to 30 hours) to provide controlled and sustained prodrug release. These delivery platforms can provide an essentially zero order delivery profile as opposed to the spiked profiles of immediate release formulations.

In another embodiment, the dosage form comprises another osmotic dosage form comprising a wall surrounding a compartment, the wall comprising a semipermeable polymeric composition permeable to the passage of fluid and substantially impermeable to the passage of prodrug present in the compartment, a prodrug-containing layer composition in the compartment, a hydrogel push layer composition in the compartment comprising an osmotic formulation for imbibing and absorbing fluid for expanding in size for pushing the prodrug composition layer from the dosage form, and at least one passageway in the wall for releasing the prodrug composition. The method delivers the prodrug by imbibing fluid through the semipermeable wall at a fluid imbibing rate determined by the permeability of the semipermeable wall and the osmotic pressure across the semipermeable wall causing the push layer to expand, thereby delivering the prodrug from the dosage form through the exit passageway to a patient over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer comprises 0.0 mg to 350 mg, in present manufacture; 0.1 mg to 250 mg of a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose) in present manufacture; 1 mg to 50 mg of an osmagent selected from the group consisting of sodium chloride, potassium chloride, potassium acid phosphate, tartaric acid, citric acid, raffinose, magnesium sulfate, magnesium chloride, urea, inositol, sucrose, glucose and sorbitol; 0 to 5 mg of a colorant, such as ferric oxide; 0 mg to 30 mg, in a present manufacture, 0.1 mg to 30 mg of a hydroxypropylalkylcellulose of 9,000 to 225,000 average-number molecular weight, selected from the group consisting of hydroxypropylethylcellulose, hydroxypropypentylcellulose, hydroxypropylmethylcellulose, and hydropropylbutylcellulose; 0.00 to 1.5 mg of an antioxidant selected from the group consisting of ascorbic acid, butylated hydroxyanisole, butylated hydroxyquinone, butylhydroxyanisole, hydroxycomarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine; and 0.0 mg to 7 mg of a lubricant selected from the group consisting of calcium stearate, magnesium stearate, zinc stearate, magnesium oleate, calcium palmitate, sodium suberate, potassium laurate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid.

In the osmotic dosage forms, the semipermeable wall comprises a composition that is permeable to the passage of fluid and impermeable to the passage of prodrug. The wall is nontoxic and it comprises a polymer selected from the group consisting of a cellulose acylate, cellulose diacylate, cellulose triacylate, cellulose acetate, cellulose diacetate and cellulose triacetate. The wall comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether selected from the group consisting of hydroxypropylcellulose or a hydroxypropylalkycellulose such as hydroxypropylmethylcellulose. The total weight percent of all components comprising the wall is equal to 100 wt %. The internal compartment comprises the prodrug-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of prodrug to a patient over time. The dosage form comprises a passageway in the wall that connects the exterior of the dosage form with the internal compartment. The osmotic powered dosage form can be made to deliver prodrug from the dosage form to the patient at a zero order rate of release over a period of up to about 24 hours.

The expression “passageway” as used herein comprises means and methods suitable for the metered release of the prodrug from the compartment of the dosage form. The exit means comprises at least one passageway, including orifice, bore, aperture, pore, porous element, hollow fiber, capillary tube, channel, porous overlay, or porous element that provides for the osmotic controlled release of prodrug. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of prodrug from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in Theeuwes et al., U.S. Pat. No. 3,845,770; Theeuwes et al., U.S. Pat. No. 3,916,899; Saunders et al., U.S. Pat. No. 4,063,064; Theeuwes et al., U.S. Pat. No. 4,088,864 and Ayer et al., U.S. Pat. No. 4,816,263. Passageways formed by leaching are disclosed in Ayer et al., U.S. Pat. No. 4,200,098 and Ayer et al., U.S. Pat. No. 4,285,987.

Regardless of the specific form of sustained release oral dosage form that is used, the prodrug is preferably released from the dosage form over a period of at least about 6 hours, more preferably, over a period of at least about 8 hours, and most preferably, over a period of at least about 12 hours. Further, the dosage form preferably releases from 0 to 20% of the prodrug in 0 to 2 hours, from 20 to 50% of the prodrug in 2 to 12 hours, from 50 to 85% of the prodrug in 3 to 20 hours and greater than 75% of the prodrug in 5 to 18 hours. Further, the sustained release oral dosage form further provides a concentration of the prodrug in the blood plasma of the patient over time, which has an area under the curve (AUC) that is proportional to the dose of the prodrug administered, and a maximum concentration Cₘₐₓ. The Cₘₐₓ is less than 75%, and is preferably, less than 60%, of the Cₘₐₓ obtained from administering an equivalent dose of the prodrug from an immediate release oral dosage form, and the AUC is substantially the same as the AUC obtained from administering an equivalent dose of the prodrug from an immediate release oral dosage form.

Preferably, the dosage forms of the invention are administered twice per day (more preferably, once per day).

### 4.8 Methods of Administration and Doses

Methods for treatment of hot flashes require administration of a GABA analog prodrug, or a pharmaceutical composition containing a GABA analog prodrug, to a patient in need of such treatment. The compounds and/or pharmaceutical compositions thereof are preferably administered orally. The compounds and/or pharmaceutical compositions thereof may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or pharmaceutical composition thereof. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. Preferably, the compounds and/or pharmaceutical compositions thereof are delivered via sustained release dosage forms, more preferably, via oral sustained release dosage forms.

The amount of GABA analog prodrug that will be effective in the treatment of hot flashes (whether hormonally, surgically, drug, or otherwise induced) in a patient will depend on the specific nature of the condition, and can be determined by standard clinical techniques known in the art. In addition, in vitro or in vivo assays may be optionally employed to help identify optimal dosage ranges. The amount of a prodrug administered will, of course, be dependent on, among other factors, the subject being treated, the weight of the subject, the severity of the affliction, the manner of administration and the judgment of the prescribing physician.

Preferably, the dosage forms are adapted to be administered to a patient no more than twice per day, more preferably, only once per day. Dosing may be provided alone or in combination with other drugs and may continue as long as required for effective treatment of the hot flashes.

Suitable dosage ranges for oral administration are dependent on the potency of the particular GABA analog drug (once cleaved from the promoiety), but are generally about 0.1 mg to about 200 mg of drug per kilogram body weight, more preferably about 1 to about 100 mg/kg-body wt. per day. Preferably, the GABA analog prodrug is a prodrug of gabapentin or pregabalin. When the GABA analog is gabapentin, typical daily doses of the drug in adult patients are 300 mg/day to 3600 mg/day and the dose of gabapentin prodrug may be adjusted to provide an equivalent molar quantity of gabapentin. Other GABA analogs may be more potent than gabapentin and lower doses may be appropriate for both the cleaved drug and any prodrug (measured on an equivalent molar basis). For example, typical doses for pregabalin in the range of 100 mg/day to 1200 mg/day are appropriate. Dosage ranges may be readily determined by methods known to the skilled artisan.

### 4.9 Combination Therapy

In certain embodiments, GABA analog prodrugs and/or pharmaceutical compositions thereof can be used in combination therapy with at least one other therapeutic agent which may be a different GABA analog prodrug. The GABA analog prodrug and/or pharmaceutical composition thereof and the therapeutic agent can act additively or, more preferably, synergistically. In one embodiment, a GABA analog prodrugs and/or a pharmaceutical composition thereof is administered concurrently with the administration of another therapeutic agent. In another embodiment, GABA analog prodrugs and/or pharmaceutical composition thereof is administered prior or subsequent to administration of another therapeutic agent.

### 5. EXAMPLES

The invention is further defined by reference to the following examples, which describe in detail, preparation of sustained release dosage form and methods for using GABA analog prodrugs to treat or prevent hot flashes. It will be apparent to those skilled in the art that many modifications, both to materials and methods, may be practiced without departing from the scope of the invention.

### 5.1 Administration of 1-{[(α-Isobutanoyloxyethoxy)carbonyl]-aminomethyl}-1-Cyclohexane Acetic Acid to Postmenopausal Women for the Treatment of Hot Flashes

Twenty postmenopausal women who have been experiencing hot flashes (an average of at least 6 per day, range 6-20 per day) over the past 12 months and who have not been treated with hormone therapy (i.e., no estrogen, progestin, tamoxifen or leuprolide therapy) over the past 2 months are recruited to an open label clinical study on the effect of administration of a gabapentin prodrug on the frequency and severity of hot flash symptoms. After a two week baseline screening assessment, the prodrug 1-{[(α-isobutanoyloxyethoxy)carbonyl]aminomethyl}-1-cyclohexane acetic acid (synthesized as described by Gallop et al., International Publication No. WO 02/100347), formulated as an immediate release dosage form in 300 mg capsules, is administered in two capsules three times daily (1800 mg/day, equal to ˜900 mg gabapentin equivalents/day) for two weeks. Each patient records the frequency and severity of hot flashes in a diary following the protocol of Guttuso et al., Obstet. Gynecol. 2003, 101, 337-345. Daily hot flash frequency is calculated by totaling the number of hot flashes per week and dividing by the number of days in the week for which data is recorded. The primary outcome measure is the percentage change in hot flash frequency from baseline to the end of treatment week two. A decrease in mean hot flash intensity of more than 35% from baseline is apparent in the treated patients, indicating the efficacy of this gabapentin prodrug in treating hot flashes in postmenopausal women.

### 5.2 Preparation of a Sustained Release Oral Dosage Form of 1-{[(α-Isobutanoyloxyethoxy)carbonyl]aminomethyl}-1-Cyclohexane Acetic Acid

A sustained release oral osmotic delivery dosage form containing the gabapentin prodrug 1-{[(α-isobutanoyloxyethoxy)carbonyl]aminomethyl}-1-cyclohexane acetic acid is prepared following methods described in Ayer et al., U.S. Pat. No. 5,707,663. Accordingly, 660 grams of 1-{[α-isobutanoyloxyethoxy)carbonyl]aminomethyl}-1-cyclohexane acetic acid and 30 grams of pharmaceutical acceptable poly(ethylene oxide), 5,000,000 molecular weight, is added to the bowl of a fluid bed granulator. The microencapsulation process is computerized and atomized in cycles. The process is initiated by first fluidizing the dry drug and the polymer powder for 3 minutes and the blended granules are microencapsulated with aqueous hydroxypropylmethylcellulose solution. The polymer solution is prepared by dissolving 35 grams of hydroxypropylmethylcellulose comprising 11,200 molecular weight in 400 grams of water. The operating conditions are as follows: spray rate of 50 grams/min/nozzle (2 nozzles are used), inlet temperature 50° C.; outlet temperature 37° C. and process air flow of 400 ft³/minute. During the coating process, the filter bag is shaken for 10 seconds after every 15 seconds of solution spraying to remove any uncoated materials. A total of 270 grams of solution is applied. After solution spraying, the microencapsulated powder is dried in the granulator to reach a moisture content of 0.25%. The dried granulation is then passed through a 16 mesh screen. Next, a total of 5.3 grams of magnesium stearate is weighed out, screened through a 40 mesh screen, and blended into the granulation using a V-blender for 2 minutes. The granulation is stored in a tightly closed bag with desiccants.

The osmotic displacement-push composition is then prepared as follows: first, 3.7 kg of sodium chloride and 150 grams of red ferric oxide are separately screened through an 8 mesh screen using a Quadro comil. Then the screened ingredients plus 7.6 kg of pharmaceutical acceptable grade poly(ethylene oxide) (7,500,000 molecular weight) and 250 grams of hydroxypropylmethylcellulose (11,200 molecular weight) are dispensed into the bowl of a Glatt fluid bed granulator. Next, the dry powders are air suspended and mixed for 3 minutes. To prepare the binder solution 420 grams of hydroxypropylmethylcellulose (11,200 molecular weight) is dissolved in 4.85 kg of water and 9.4 grams of butylated hydroxytoluene is dissolved in 60 grams of denatured ethanol. The two solutions are combined and mixed to form the final binder solution. The conditions monitored during the process are as follows: solution spray rate of 400 g/min (3 nozzles are used); inlet temperature 45° C.; outlet temperature 24° C. and process air flow of 1,500 ft³/minute. The granulating process is computerized and automated in cycles. Each cycle contains 1.5 minutes of solution spraying followed by 10 seconds of bag shaking to remove any possible powder deposits. A total of 4.4 kg of solution is sprayed. After solution spraying, the granulated particles are dried in the granulator for 50 minutes at 21° C. to reach a moisture content of 0.3%. The granules are removed and sized through an 8 mesh screen. Then 28 grams of magnesium stearate, screened through a 16 mesh screen, is mixed into the granulation using a tumbler for 3 minutes at 8 rpm.

Next, the 1-{[(α-isobutanoyloxyethoxy)carbonyl]aminomethyl}-1-cyclohexane acetic acid drug composition and the push composition are compressed using a tablet press into bilayer cores of tablet shape as follows: first 700 mg of 1-{[(α-isobutanoyloxyethoxy)carbonyl]aminomethyl}-1-cyclohexane acetic acid drug composition is added to a punch and lightly precompressed, then 421 mg of the push composition is added and the layers are pressed under a pressure head of 1.5 ton (3000 lbs) into a 0.75″ length modified capsule contacting layered arrangement. The compression process is done in a humidity controlled environment. The relative humidity during the process is 35% RH (relative humidity) or lower. The compressed cores are stored in a tightly closed bag with desiccants.

The bilayered arrangements next are coated with a semipermeable wall. The wall-forming composition comprises 100% cellulose acetate having a ˜40% acetyl content. The polymer is dissolved in 100% acetone to make a 4% solid solution. The wall forming composition is sprayed at 26 grams/min onto and around the bilayer cores in a tablet coater until a dry weight of 90 mg/core is achieved.

Next, one 10 mil (0.254 mm) exit passageway is mechanically drilled through the semipermeable wall to connect the drug layer with the exterior of the dosage system. The residual solvent is removed by first drying for 120 hours at 50° C. and 30% relative humidity, then the systems are dried for 2 hours at 50° C. to remove excess moisture. The drug dosage form produced by this process provides: ˜90 wt % 1-{[(α-isobutanoyloxyethoxy)carbonyl]aminomethyl}-1-cyclohexane acetic acid, 4 wt % hydroxypropylmethylcellulose (11,200 molecular weight), 4 wt % poly(ethylene oxide) (5,000,000 molecular weight) and 1 wt % magnesium stearate in the drug layer. The push composition comprises 63.7 wt % poly(ethylene oxide) (7,500,000 molecular weight), 30 wt % sodium chloride, 5 wt % hydroxypropylmethylcellulose (11,200 molecular weight), 1 wt % red ferric oxide, 0.25 wt % magnesium stearate, and 0.075 wt % of butylated hydroxytoluene. The wall is 100 wt % cellulose acetate having a ˜40% acetyl content. The dosage form has one passageway, 10 mils (0.254 mm), and it has a 1-{[(α-isobutanoyloxyethoxy)carbonyl]aminomethyl}-1-cyclohexane acetic acid release rate of >20 mg/hr and a half life for drug release of >8 hours in artificial gastric fluid.

### 5.3 Treatment of Hot Flashes in Postmenopausal Women by Administration of 1-{[(α-Isobutanoyloxyethoxy)carbonyl]-aminomethyl}-1-Cyclohexane Acetic Acid via a Sustained Release Oral Dosage Form

Twenty postmenopausal women who have been experiencing hot flashes (an average of at least 6 per day, range 6-20 per day) over the past 12 months and who have not been treated with hormone therapy (i.e., no estrogen, progestin, tamoxifen or leuprolide therapy) over the past 2 months are recruited to an open label clinical study on the effect of administration of gabapentin prodrugs on the frequency and severity of hot flash symptoms. After a two week baseline screening assessment, the prodrug 1-{[(α-isobutanoyloxyethoxy)carbonyl]aminomethyl}-1-cyclohexane acetic acid, formulated as osmotic sustained release capsules containing 700 mg drug (preparation of the sustained release capsules is described in Section 5.2 above), is administered in two capsules twice daily (2800 mg/day, equal to ˜1400 mg gabapentin equivalents/day) for two weeks. Each patient records the frequency and severity of hot flashes in a diary following the protocol of Guttuso et al., Obstet. Gynecol 2003, 101, 337-345. Daily hot flash frequency is calculated by totaling the number of hot flashes per week and dividing by the number of days in the week for which data is recorded. The primary outcome measure is the percentage change in hot flash frequency from baseline to the end of treatment week two. A decrease in mean hot flash intensity of more than 35% from baseline is apparent in the treated patients, indicating that delivery of a gabapentin prodrug from a sustained release oral dosage form is efficacious in treating hot flashes in postmenopausal women.

It will be apparent to those skilled in the art that many modifications, both to materials and methods, may be practiced without departing from the scope of this disclosure. Accordingly, the present embodiments are to be considered as illustrative and not restrictive and the invention is not to be limited to the details given herein, but may be modified within the scope and equivalents of the appended claims.

All publications and patents cited herein are incorporated by reference in their entirety.

1. A method of treating or preventing hot flashes in a patient comprising administering to the patient in need of such treatment or prevention a therapeutically effective amount of a prodrug of a GABA analog, or a pharmaceutically acceptable salt, hydrate or solvate thereof.

2. A method of treating or preventing hot flashes in a patient comprising administering to the patient in need of such treatment or prevention a pharmaceutical composition comprising a therapeutically effective amount of a prodrug of a GABA analog, or a pharmaceutically acceptable salt, hydrate or solvate thereof and a pharmaceutically acceptable vehicle.

3. The method of claim 1 or claim 2, wherein the GABA analog is gabapentin or pregabalin.

4 The method of claim 3, wherein the GABA analog is administered in an amount of between about 10 mg to about 5000 mg per day.

5. The method of claim 1, wherein the patient is a female patient.

6. The method of claim 5, wherein the female patient is postmenopausal.

7. The method of claim 6, wherein menopause is drug induced or surgically induced.

8. The method of claim 1, wherein the patient is a male patient.

9. The method of claim 5 or claim 8, wherein the hot flashes are drug-induced.

10. The method of claim 1 or claim 2, wherein the prodrug is administered orally, parenterally, subcutaneously, intravenously, intramuscularly, intraperitoneally, intranasally instillationally, intracavitarally or intravesical instillationally, intraocularly, intraarterially, intralesionally, by implantation or by application to mucous membranes.

11. The method of claim 1 or claim 2, wherein the prodrug is administered orally.

12. The method of claim 1 or claim 2, comprising administering the prodrug in a sustained release oral dosage form.

13. The method of claim 12, wherein the dosage form releases the prodrug gradually over a period of at least about 6 hours after swallowing the dosage form, thereby providing a therapeutic concentration of a GABA analog in the plasma of the patient.

14. The method of claim 12, wherein the dosage form is an osmotic dosage form, a prodrug-releasing polymer, a prodrug-releasing lipid, a prodrug-releasing wax, tiny timed-release pills or prodrug releasing beads.

15. The method of claim 1 or claim 2, wherein the prodrug of a GABA analog has the structure of Formula (I): 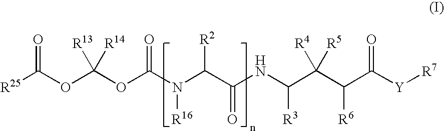
or a pharmaceutically acceptable salt, hydrate or solvate thereof, wherein:
n is 0 or 1;
Y is O or S;
R¹⁶ is hydrogen, alkyl or substituted alkyl;
R² is selected from the group consisting of hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, or optionally, R² and R¹⁶ together with the atoms to which they are attached form a cycloheteroalkyl or substituted cycloheteroalkyl ring;
R³ and R⁶ are independently selected from the group consisting of hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl and substituted heteroarylalkyl;
R⁴ and R⁵ are independently selected from the group consisting of hydrogen, alkyl, substituted alkyl, acyl, substituted acyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl and substituted heteroarylalkyl or optionally, R⁴ and R⁵ together with the carbon atom to which they are attached form a cycloalkyl, substituted cycloalkyl, cycloheteroalkyl, substituted cycloheteroalkyl or bridged cycloalkyl ring;
R⁷ is selected from the group consisting of hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl and substituted heteroarylalkyl;
R¹³ and R¹⁴ are each independently hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroaryl, substituted heteroaryl, heteroarylalkyl or substituted heteroarylalkyl or optionally, R¹³ and R¹⁴ together with the carbon atom to which they are attached form a cycloalkyl, substituted cycloalkyl, cycloheteroalkyl or substituted cycloheteroalkyl ring; and
R²⁵ is selected from the group consisting of acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl and substituted heteroarylalkyl.

16. The method of claim 15, wherein the prodrug of a GABA analog has the structure of Formulae (II) or (III):

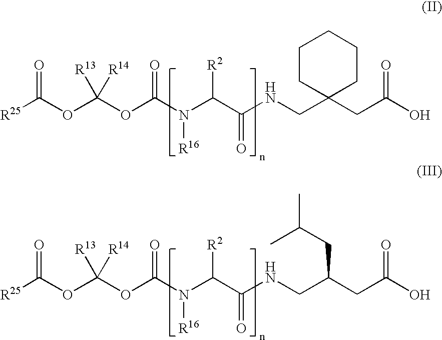

17. The method of claim 16, wherein n is 0.

18. The method of claim 16, wherein n is 1, R¹⁶ is hydrogen and R² is selected from the group consisting of hydrogen, methyl, 2-propyl, 2-butyl, isobutyl, tert-butyl, cyclopentyl, cyclohexyl, phenyl, benzyl, 4-hydroxybenzyl, 4-imidazolylmethyl, 3-indolylmethyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂(CH₂)₃NH₂ and —CH₂CH₂CH₂NHC(NH)NH₂.

19. The method of claim 17, wherein R²⁵ is selected from the group consisting of methyl, ethyl, propyl, isopropyl, butyl, isobutyl and sec-butyl, R¹³ is methyl and R¹⁴ is hydrogen.

20. The method of claim 17, wherein R²⁵ is isopropyl, R¹³ is methyl and R¹⁴ is hydrogen.

21. A pharmaceutical composition for treating a patient suffering from hot flashes comprising a therapeutically effective amount of a prodrug of a GABA analog or a pharmaceutically acceptable salt, hydrate or solvate thereof, and a pharmaceutically acceptable vehicle.

22. A pharmaceutical composition for preventing hot flashes in a patient at risk of hot flashes comprising a therapeutically effective amount of a prodrug of a GABA analog or a pharmaceutically acceptable salt, hydrate or solvate thereof and a pharmaceutically acceptable vehicle.
